(19) United States
(12) Patent Application Publication
(10) Pub. No.: US 2006/0292208 A1
(43) Pub. Date: Dec. 28, 2006

(54) Sulfonated styrene polymers for medical articles and barrier web constructs

(75) Inventor: David J. Vachon, Spokane, WA (US)

Correspondence Address: HESLIN ROTHENBERG FARLEY & MESITI PC, 5 COLUMBIA CIRCLE, ALBANY, NY, 12203, US

(73) Assignee: Aegis Biosciences, LLC, Palm Harbor, FL (US)

(21) Appl. No.: 11/180,483

(22) Filed: Jul. 12, 2005

Related U.S. Application Data
(60) Provisional application No. 60/586,927, filed on Jul. 12, 2004.

(51) Int. Cl. A61L 15/00

(52) U.S. Cl. 424/445

(57) ABSTRACT

Sulfonated styrene copolymer compositions and salts thereof, and their use in drug delivery devices, such as wound dressings, as well as their use for controlling chemical agents and for controlling biological organisms are presented.

### CROSS-REFERENCE TO RELATED APPLICATIONS

This application claims priority from U.S. Provisional Application Ser. No. 60/586,927, filed Jul. 12, 2004, the entire contents of which are incorporated herein by reference.

### FIELD OF THE INVENTION

The invention relates to sulfonated styrene copolymer compositions and salts thereof, and their use in drug delivery devices, such as wound dressings, as well as their use for controlling chemical agents and for controlling biological organisms.

### BACKGROUND OF THE INVENTION

Research has established that healing of wounds such as burns, skin ulcers, pressure sores and traumatic injuries is facilitated when the wound bed is kept moist and clean. Wound dressings are particularly useful for this purpose and have become an accepted therapy for treating wounds.

Several types of wound dressings are commercially available, including hydrogels, hydrocolloids, semi-permeable adhesive films, perforated films, alginates, polysaccharide beads, and polyurethane foams. Additionally, wound dressings have been used as carriers for the delivery of therapeutic agents for treatment of the wound. Unfortunately, the use of hydrogels, as well as other wound dressing products, as carriers for therapeutic agents has been severely limited by the composition and resulting physical properties of available products.

Many of the commercial moist wound dressings are composed of cross-linked ethylene oxide polymers. These dressings are typically manufactured by single step sterilization and irradiation of the polymer, resulting in a sheet of insoluble water-swollen gel. Very few therapeutic agents to be incorporated prior to the cross-linking step are stable enough to withstand this high-energy radiation. Alternative methods include dehydrating the gel following cross-linking and rehydrating with an aqueous solution of the therapeutic agent. However, dressings produced by this method frequently develop unacceptable cosmetic defects when dehydrated and rehydrated.

A need exists for polymer compositions, especially those carrying therapeutic agents, as well methods of use and production processes that overcome at least one of the aforementioned deficiencies.

U.S. Pat. No. 5,840,387 to Berlowitz-Tarrant et al. discloses use of a sulfonated copolymer of styrene for delivery of therapeutic agents and U.S. Pat. No. 6,306,419 to Vachon et al. discloses the use of a sulfonated copolymer for use as a hydrogel wound dressing with controlled release capability. The entire contents of each are incorporated herein by reference and all of the references therein are incorporated herein by reference.

### SUMMARY OF THE INVENTION

An aspect of the present invention relates to a composition comprising: a substrate; a polymer composition disposed on at least a first side of said substrate, wherein said polymer composition comprises at least two salts of a sulfonated styrene copolymer having a chemical controlling agent and/or a biological organism controlling agent as a counter ion.

A second aspect of the present invention relates to a wound dressing comprising: at least a first layer comprising a first salt of a sulfonated styrene copolymer having at least one surface contactable with a wound and disposed on said one surface a second salt of said sulfonated styrene copolymer, wherein said second salt is disposed on said one surface in a preselected pattern.

A third aspect of the present invention relates to a wound dressing comprising: at least a first layer impregnated with at least two salts of a sulfonated styrene copolymer having at least one surface contactable with a wound, wherein said at least two salts are impregnated in said first layer in a preselected pattern.

A fourth aspect of the present invention relates to a method for controlling chemical agents and/or biological organisms comprising: providing a composition comprising: i) a substrate and ii) a polymer composition disposed on at least a first side of said substrate, wherein said polymer composition comprises at least two salts of a sulfonated styrene copolymer having a chemical controlling agent and/or a biological organism controlling agent as a counter ion; and exposing said composition to a chemical agent and/or a biological organism wherein said chemical controlling agent and/or said biological organism controlling agent interacts with a chemical agent and/or a biological organism.

A fifth aspect of the present invention relates to a process for patterning a wound dressing comprising: providing a substrate; applying a sulfonated styrene copolymer to said substrate to form a wound dressing; applying a first salt onto said wound dressing in a preselected pattern; and applying a second salt onto said wound dressing in a preselected pattern.

All references, patents and patent applications cited herein are hereby incorporated by reference in their entirety.

### BRIEF DESCRIPTION OF THE DRAWINGS

FIG. 1 depicts a first wound dressing, in accordance with the present invention; and

FIG. 2 depicts a second wound dressing, in accordance with the present invention.

### DETAILED DESCRIPTION OF THE INVENTION

The present invention relates to sulfonated styrene copolymers and salts thereof used in the production of compositions such as coatings and webs that are useful in the control and neutralization of chemical agents and biological organisms (chemical & biological warfare agents, and biological organisms in laboratory & hospital environments), and the control of biological processes (coagulation, protease inhibition, bacterial colonization, and cellular proliferation).

Sulfonated styrene polymers suitable for use in the compositions, methods, processes, and wound dressings of the present invention are described in U.S. Pat. Nos. 5,468,574; 5,679,482; 5,677,074; 6,110,616; 6,413,298; 6,383,391; 6,841,601; U.S. Published Application No. 20030106680; U.S. application Ser. No. 11/033,461 and WO2005/030812, to Dais Analytic Corporation, and in U.S. Pat. Nos. 5,840,387 and 6,306,419 and U.S. Published Application No. 2004/0142910, to Aegis Biosciences LLC, the entire contents of all of which are incorporated herein by reference.

A composition comprising: a substrate; and a polymer composition disposed on at least a first side of the substrate, wherein the polymer composition comprises at least two salts of a sulfonated styrene copolymer is presented in accordance with the present invention. In an embodiment of the present invention, the salts of the sulfonated styrene copolymer have a chemical controlling agent and/or a biological organism controlling agent as a counter ion

Substrates that may be used to support the polymer composition include but are not limited to a solid film, a perforated film, a woven fabric, a non-woven fabric, a knit fabric, a latex material, a porous material, a non-porous material, a polymeric material, an article of wear, and the like.

A solid film refers to a thin sheet of uniform gauge without any underlying support. In polymer science, it is a term for sheeting having a nominal thickness not greater than 0.25 mm (0.01 inches) (ASTM D883-91a Standard Terminology Relating to Plastics). Examples of a film include but are not limited to a vinyl film, a nylon film, a spandex film, a saran film, a poly(chlorotrifluoro)ethylene film, a polyethylene film, a polypropylene film, a polystyrene film, an ethylene-vinylacetate film, a styrene-alkylene film, and the like. Perforated films are films as previously described having a hole, a gap, or series of holes or gaps punched or bored through the film.

A woven fabric refers to a natural or synthetic textile material formed on a loom with two separate yarns (warp and filling) that are at right angles to each other. The two yarns go over and under each other in a designated pattern. Examples of a woven fabric include but are not limited to a polyester fabric, a poly(ethylene terephthalate) fabric, a rayon fabric, an acrylic fabric, polymeric fabrics, cotton, jute, silk, wool, linen, twill, toile, bunting, duck, faille, gabardine, herringbone, jacquard, muslin, lawn, leno, and other like fabric weaves.

A non-woven fabric refers to a natural or synthetic textile material formed by laying a continuous web of random spaced fibers to form a uniform batting. The fibers are then bonded to form a fabric by chemical adhesion, thermal or mechanical processes. Examples of a non-woven fabric include but are not limited to a glass fabric, a polypropylene, a bicomponent fabric (bico), pulp, and the like. A knitted fabric refers to natural or synthetic textile material formed by a single or multiple yarns making interlocking loops. Examples of a knitted fabric include but are not limited to tricot, jersey, percaile, balbriggan, and the like.

A latex material generally refers to a material formed from a dispersion of resinous polymer in a mainly aqueous vehicle. Examples of latex include but are not limited to materials arising from aqueous emulsions containing at least one of styrene-alkylene, styrene-acrylics, or blends thereof. The aforementioned aqueous emulsion may or may not contain another polymer system therein having but not limited to an acrylic, silicone, polyurethane, polyester, polyamide, ethylene-vinyl-acetate, or epoxy system, and the like.

A porous material refers to a generally uniform and/or homogeneous material having pores that allow for the passage of a gas and/or a fluid through the pores of the material as wells as any thing being carried by the gas or fluid. Examples of a porous material include but are not limited to a gas filtration membrane or a liquid filtration membrane comprising a polyamide, a polyurethane, a polycarbonate, a polyester, a poly(tetrafluoroethylene), a polysulfone, and the like.

A non-porous material refers to a generally uniform and/or homogeneous material not having pores thus preventing the passage of a gas and/or a fluid through the material as well as any thing being carried by the gas or fluid. Examples of a non-porous material include but are not limited to films or materials created by extrusion, press melting, casting, and the like.

A polymeric material refers to a material fully comprising, partially comprising, fully coated, or partially coated with a polymer. Typically the polymer as used herein is a macromolecule comprising of multiple repeating smaller units or molecules (monomers) derived, actually or conceptually, from smaller units or molecules. The polymer may be natural or synthetic. Examples of a polymeric material include but are not limited to a polymer solid, a polymer film, a polymer bead, a polymer nanobead, a polymer microsphere, a polymer fiber, a polymer weave, a polymer knit, and the like.

An article of wear refers to any individual thing or element belonging to the class of clothing. Examples include but are not limited to a shirt, a jacket, a pant, a sneaker, a boot, socks, a hat, a bodysuit, a hood, gloves, hazardous material protective clothing, goggles, and the like.

The polymer composition of the present invention comprises at least two salts of a sulfonated styrene copolymer. The term “sulfonated styrene copolymer” as used herein refers to a polymer having residues derived from a styrene monomer, the aromatic ring of which is substituted with up to at least one sulfonate group per aromatic ring. The term encompasses homopolymers containing residues derivable from the sulfonated styrene and copolymers containing residues derivable from styrene and the sulfonated styrene, as well as copolymers containing residues of other comonomers in addition to styrene and the sulfonated styrene.

The sulfonated styrene copolymer of the polymer composition contains at least one structural unit of the first salt of the sulfonated styrene copolymer or the second salt of the sulfonated styrene copolymer as represented by formula 1:

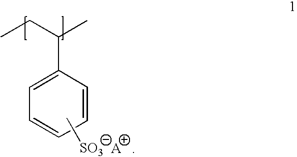

Other comonomers that may be used an embodiment of the present invention include any alkylene monomer that may be polymerized with styrene such as monomers derived from ethylene, propylene, acrylate, acrylamide, isobutylene, and any other monomers having a double bond functionality that are known to polymerize by free radical polymerization, cationic polymerization, or anionic polymerization.

Typically the sulfonated styrene copolymer is selected from the group consisting of a sulfonated styrene-ethylene-butylene-styrene triblock copolymer, a sulfonated styrene-ethylene copolymer, a sulfonated stryrene-acrylate copolymer, a sulfonated styrene-isobutylene-styrene copolymer, a sulfonated styrene-divinylbenzene copolymer, and combinations thereof.

The sulfonated styrene copolymer may additionally comprise residues derived from at least one olefin comonomer. Preferred olefin comonomers include monoolefins, such as ethylene, propylene, butylene, and isobutylene, and also diolefin monomers, such as butadiene and isoprene. Other comonomers, such as acrylate monomers, may be used, provided that the properties of the copolymer are sufficient for the end use. Homopolymers of styrene may be sulfonated to produce a copolymer containing residues derivable from styrene and the sulfonated styrene.

When an unsulfonated styrene copolymer contains residues derived from a diolefin comonomer, such as butadiene, a residual alkene functionality is usually present in the copolymer. In this case, the copolymer may be hydrogenated in order to reduce the double bonds prior to the sulfonation step. The resulting copolymer is referred to as a reduced or hydrogenated copolymer. The copolymer may be hydrogenated by methods known in the art, for example, by hydrogen gas in the presence of catalysts such as Raney Nickel, platinum or palladium. Hydrogenated statistical copolymers of styrene and butadiene are also commercially available from a number of sources that include Kraton Polymers Inc. and the Japanese manufacturer Kaneka.

Unsulfonated styrene-ethylene-butylene-styrene triblock copolymers may be obtained from Shell as the Kraton® series. The styrene content of the Kraton® copolymers is typically about 30% before sulfonation. Unsulfonated rubbery styrene butadiene copolymers, known as styrene butadiene rubber (SBR) are commercially available from Goodyear. Unsulfonated styrene ethylene copolymers may be obtained from Dow Chemical. Sulfonated styrene copolymers may be obtained from Dais Analytic Corporation or from Dow Chemical in the form of Dowex® ion exchange resins.

The non-cross linked sulfonated styrene copolymer(s) may be readily blended with other polymers. These blends, depending on the amount of each polymer and the thermodynamics of mixing, can afford polymeric materials ranging from phase-separated to single phase. An advantage of blending is that selected properties of the individual components may be obtained in the resulting material. Block copolymers having both components of the blend in a single chain may be used to the increase the compatibility of the blend components.

Several types of styrene-containing polymers are commercially available, including statistical, block, and graft copolymers, and combinations of these types. The term “statistical” is well known in the art, and refers to copolymers that are synthesized by methods that are not designed to produce blocks or grafts in the copolymer. This type of polymer is also commonly referred to as a random copolymer. The monomers polymerize according to their relative reactivities. Any of these types of polymers may be used. Particularly preferred sulfonated styrene copolymers are sulfonated styrene-ethylene-butylene-styrene triblock copolymers, statistical sulfonated styrene butadiene copolymers, sulfonated styrene-isobutylene-styrene triblock copolymers, sulfonated styrene-acrylate-styrene copolymers, sulfonated statistical styrene ethylene copolymers, and sulfonated statistical styrene-acrylate copolymers.

The sulfonated styrene copolymer typically is a mixture of at least 25% of the sulfonated styrene copolymer and some other component, such as blends of unsulfonated styrene block copolymers with sulfonated styrene block copolymers. The other component may be selected from a homopolymer, another copolymer, a block polymer, or a vulcanizable species. Vulcanizable species that may be used include but are not limited to an acrylate, a silane, a diallyl an ester, an isocyanate, a maleimide, 1,2 polybutadiene, a divinyl benzene, an itaconic acid, an allylamine, a vinylamine, a N-vinylcarbazole, an aminostyrene, a maleic anhydride and the like.

Homopolymers that may be used include but are not limited to a polystyrene, a polyethylene, a polypropylene, a polydimethylsiloxane, a polybutadiene, a polyisoprene, and the like. Copolymers that may be used include but are not limited to a polyurethane, a polyester, a polyimide, a polyamide, an ethylene-propylene polymer, a silicone rubber, a latex rubber, and a butyl rubber. Block polymers that may be used include but are not limited the group consisting of styrene alkylene copolymers comprising acrylonitrile-butadiene-stryene (ABS), styrene-acrylonitrile (SAN), sulfonated styrene-ethylene-butylene-styrene (SEBS), sulfonated styrene-ethylene-propylene-styrene (SEPS), hydrogenated sulfonated styrene-isoprene-styrene, sulfonated styrene-isobutylene-styrene (SIBS), sulfonated styrene-ethylene-ethylene-propylene-styrene (SEEPS), sulfonated styrene-ethylene (SE), and styrene-ethylene-propylene copolymers.

In an embodiment of the present invention, the composition of the sulfonated styrene copolymers typically range from about 20% styrene to about 80% styrene. That is, the copolymer contains about 20%-80% by weight of residues derived from styrene before sulfonation of the aromatic ring of the styrene residues. The copolymer composition may be adjusted by varying the level of styrene and/or the comonomer(s) to provide the desired properties in the end product.

In an embodiment of the present invention, the level of sulfonation of the styrene residues is at least 15 mole percent and may increase up to at least 70 mole percent. However, where a blend of a styrene/sulfonated styrene copolymer with another polymer is utilized, higher levels of sulfonated styrene may be desirable. Sulfonation of the styrene residues is typically performed after completion of the polymerization. Methods for sulfonating styrene copolymers are known in the art. One suitable method is described in U.S. Pat. No. 5,468,574 to Ehrenberg et al., the entire contents of which are incorporated herein by reference. A second method is the sulfonation of hydrogenated block copolymers of styrene and butadiene to a level of about 25 mole percent as described in U.S. Pat. No. 5,239,010 to Balas et al., the entire contents of which are incorporated herein by reference.

The method of sulfonating the aromatic ring of the styrene residues where high levels of sulfonation may be achieved, is described in published PCT application, WO 99/38896, the entire contents of which are incorporated herein by reference. The application discloses the preparation of an acetyl sulfate sulfonation agent by the addition of sulfuric acid to a solution of acetic anhydride in 1,2-dichloroethane (DCE). An appropriate amount of the sulfonation agent is reacted with a styrene copolymer in a DCE solution to yield a sulfonated copolymer having a desired level of the sulfonic acid moiety.

In an embodiment of the present invention the molecular weight (MW) of the polymer preferably ranges from about 20,000 to about 1,000,000. Typically though, the MW of the polymers used are in a range from about 50,000 to about 900,000. With regard to a lower limit for molecular weight, highly sulfonated styrene polymers having a relatively low molecular weight may be water soluble. In general, it is desirable that sulfonated styrene polymer have a molecular weight sufficiently high that the polymer is not water soluble. Typically the level of sulfonation of the styrene copolymer is at least 15% mole percent and the molecular weight is at least 20,000. With regard to an upper limit for molecular weight, the molecular weight of the copolymer typically is less than about 1,000,000 in order to control the viscosity of sulfonated styrene copolymer solutions during the manufacturing process.

The sulfonated styrene copolymer of the present invention may be used in the acid form wherein the sulfonate group is —SO₃H or may be used in its respective salt form following deprotonation by a base. One structural unit of the salts of the sulfonated styrene polymer of the composition of the present invention are represented by formula 1, described supra, wherein A⁺ is a cationic counter ion. An ion refers to an atom or group of atoms that has a net electric charge by the loss or gain of electrons.

Herein the styrene sulfonate anion of the salt of the sulfonated styrene copolymer in formula 1 will be referred to as —SO₃⁻ unless specifically noted otherwise. Salt forms that are possible can be classified according to the cationic counter ion used. Classifications include chemical controlling agents and biological organism controlling agents.

Ions of chemical controlling agents include but are not limited to a Cobalt (Co) ion, a Vanadium (V) ion, a Ruthenium (Ru) ion, a Palladium (Pd) ion, a Rhodium (Rh) ion, a Manganese (Mn) ion, an Iron (Fe) ion, a Copper (Cu) ion, a Platinum (Pt) ion, an ion of chloroamine, an ion of hydrazine, an ammonium ion, an ion of an alkyl amine, an ion of an alkyl diamine, an ion of an aromatic amine, an ion of an aromatic diamine, and the like.

The ammonium ion as used in an embodiment of the present invention is NH₄⁺ wherein all substituents of the Nitrogen atom are Hydrogen atoms. Ions of all the other amines and diamines refers to structures wherein the Nitrogen atom has at least one substituent not being a Hydrogen atom.

Biological organism controlling agents can be further classified into groups comprising of ions of antibacterial agents, ions of antiviral agents, ions of antiparasitic agents, ions of antifungal agents, ions of thrombogenic agents, and the like. Ions of antibacterial agents include but are not limited to an ion of chlorhexidine, an ammonium ion, a ion of benzalkyl amine, a hexyl pyridinium ion, an ion of an alkyl amine, a Co ion, a V ion, a Ru ion, a Pd ion, a Rh ion, a Mn ion, an Fe ion, a Cu ion, a Pt ion, isoniazid, ethambutol, pyrazinamide, streptomycin, clofazimine, rifabutin, fluoroquinolones, ofloxacin, sparfloxacin, rifampin, dapsone, tetracycline, doxycyline, erythromycin, ciprofloxacin, doxycycline, ampicillinand, and the like. Ions of antiviral agents include but are not limited to an ion of acyclovir, an ion of trifluorouridine, an ion of foscarnet, an ion of ganciclovir, and the like.

Ions of antiparasitics include but are not limited to nystatin, metronidazole, tinidazole, diloxanide, pyrimethamine, clindamycin, lincomycin, azithromycin, clarithromycin, pentamidine, atovaquone, paromomycin, diclazaril, and the like. Ions of antifungal agents include but are not limited to allylamines, azoles, antimetabolites, glucan synthesis inhibitors, amphotericin B, ketoconazole, fluconazole, and the like. Ions of thrombogenic agents include but are not limited to a Calcium (Ca) ion, a Zinc (Zn) ion, a Mn ion, a Mg ion, an ion of lysine, an ion of polylysine, an ion of aminobutryic acid, an ion of aminohexanoic acid, and the like.

The amount of the cationic species of the chemical controlling agent and the biological controlling agent in salt of the polymer composition of the present invention typically is a range from about 1 percent by weight (wt %) to about 20 wt % of the styrene sulfonate copolymer composition. The ratio range of the first salt to the second salt present in the polymer composition is from about 0.1 mole % the first salt to about 99.9 mole % to about 99.9 mole % the first salt to 0.1 mole % the second salt. Alternative ratio ranges of the first salt to the second salt present in the polymer composition also include 20 mole % to about 80 mole %, 80 mole % to about 20 mole %, 30 mole % to about 70 mole %, 70 mole % to about 30 mole % respectively, and combinations thereof. The salts of the sulfonated styrene copolymers have the properties of being lipophilic, lipophobic, and varying degrees of both.

An example of a composition in accordance with the present invention is a substrate having a sulfonated styrene-alkali metal salt and a sulfonated styrene-therapeutic agent salt disposed on one side of the substrate. A poly(ethylene terephthalate) (PET) fabric was used as substrate and coated with a 60% sulfonated styrene-ethylene-butylene-styrene (SSEBS) triblock copolymer solution.

The SSEBS coated fabric then was treated with NaHCO₃ to deprotonate the sulfonated styrene unit of the SSEBS copolymer affording the first salt of the sulfonated styrene copolymer (—SO₃⁻Na⁺). The coated fabric then was treated to afford the second salt of the sulfonated styrene copolymer. Drops of a doxycycline (Dox):HCl solution (0.1 Molar (M) in deionized (DI) H₂O) were placed on the fabric having thereon the first salt in a preselected pattern (dots). The fabric was allowed to dry and then further treated with DI water to rinse away any unbound Dox.

The final result was a composition comprising a PET fabric coated with the —SO₃⁻Na⁺ salt and a —SO₃⁻Dox⁺ salt wherein the —SO₃⁻Dox⁺ salt was in a polka dot pattern across the surface of the PET fabric. Another example of the composition is a PET fabric coated with —SO₃⁻Dox⁺ salt and a —SO₃⁻Arginine⁺ salt wherein the —SO₃⁻Argenine⁺ salt was in a polka dot pattern across the surface of the PET fabric. The composition was formed as described above substituting Na with Arginine.

The above example of a composition is not meant to limit the scope of compositions in an embodiment of the present invention. Other compositions can be produced using the same process by just varying the substrate, the first salt of the sulfonated styrene copolymer, and/or the second salt of the sulfonated styrene copolymer. For example; the substrate may be a polysulfone filtration membrane, the first salt may be —SO₃⁻Na⁺, and the second salt may be —SO₃⁻Lysine⁺. Any substitution of the substrate and the counter ions of the two salts with examples described supra will result in various compositions of the present invention. For example, a composition in accordance of the present invention includes a substrate being a wound dressing.

A wound dressing comprising at least a first layer comprising a first salt of the sulfonated styrene copolymer having at least one surface contactable with a wound and disposed on the one surface a second salt of the sulfonated styrene copolymer, wherein the second salt is disposed on the one surface in a preselected pattern is presented in accordance with the present invention.

The first salt or the second salt of the sulfonated styrene copolymer has at least one structural unit represented by the formula 1: 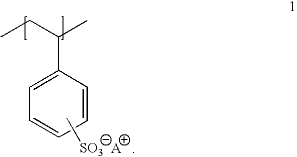
wherein A⁺ is an alkali metal ion, an alkaline earth ion, an oligodynamic metal ion, a pyridinium ion, a tetra-alkyl ammonium ion, an alkyl ammonium ion, an ion of a benzalkyl amine, an alkyl pyrazinium ion, an ion of an alkyl amine, or an ion of a chlorhexidine.

An alkali metal is a metal from Group I of the periodic table. Examples include Lithium (Li), Sodium (Na), Potassium (K), Rubidium (Rb), Cesium (Cs), and Francium (Fr). An alkaline earth metal is a metal from Group II of the periodic table. Examples include Beryllium (Be), Magnesium (Mg), Ca, Strontium (Sr), and Barium (Ba). An oligodynamic metal is a metal active or effective in very small quantities against certain germicides or heavy-metal toxins. Examples include but are not limited to Silver (Ag), Gold (Au), Pt, Pd, Mercury (Hg), Cu, Tin (Sn), Antimony (Sb), Bismuth (Bi), Zinc (Zn), Aluminum (Al), Mg, and the like.

The counter ions listed above are not meant to limit the scope of the counter ions and analogous salts or forms that may be used in an embodiment of the present invention. If the first salt has a counter ion from a group listed above, the second salt may also have any counter ion previously presented. Counter ions available for use include ions of therapeutic agents such as an antibacterial agent, an anesthetic, a growth factor, spermicide, an antiviral agent, an anti-inflammatory agent, an antihistamine, an analgesic agent, an anti-neoplastic agent, an antihistamine, a hormone, a kerolytic agent, a tranquilizer, a vitamin, a base-pair nucleotide, a cytokine, and the like.

Other counter ions available for use further include a Cobalt ion, an ion comprising Vanadium, a Ruthenium ion, a Palladium ion, an Iron ion, a Manganese ion, a Platinum ion, a Rhodium ion, a Copper ion, an ion of chloroamine, an ion of hydrazine, an ion of an akyl diamine, an ion of an aromatic amine, an ion of an aromatic diamine ion, and combinations thereof.

Typically the two salts of the sulfonated styrene copolymer are disposed on the surface of the wound dressing but also may be impregnated into the surface of the wound dressing. Impregnated is generally refers to the wound dressing that is completely or partially filled, and/or saturated with at least one of the two salts of the sulfonated styrene copolymer.

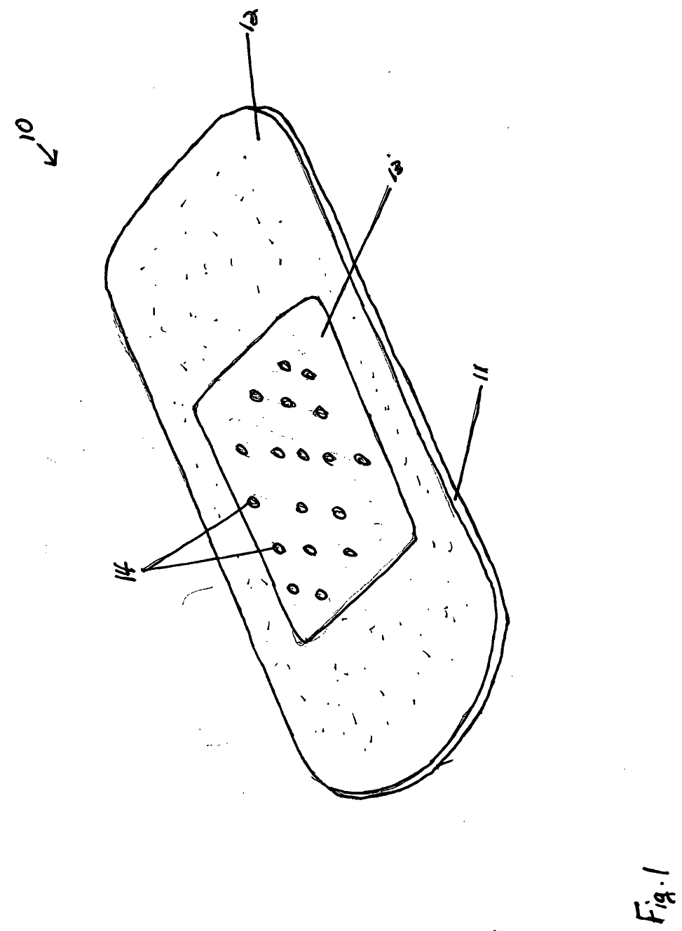

FIG. 1 depicts a wound dressing in accordance with the present invention. Referring to FIG. 1, the wound dressing 10 comprises a backing 11, an adhesive layer 12, and a sulfonated styrene copolymer layer 13. The adhesive layer 22 is coated on one side of the backing 11 and the copolymer layer 13 is affixed on a second side of the backing 12. Portions of the adhesive layer 12 are exposed and are used to secure the dressing 10 to a body or body part in the same fashion as a conventional adhesive bandage.

In an embodiment of the present invention, the copolymer layer 13 is in the form of an island pad. In this example, the copolymer layer 13 is comprised of a —SO₃⁻Na⁺ salt and a second salt 14, —SO₃⁻Dox⁺, disposed thereon in a preselected pattern of dots.

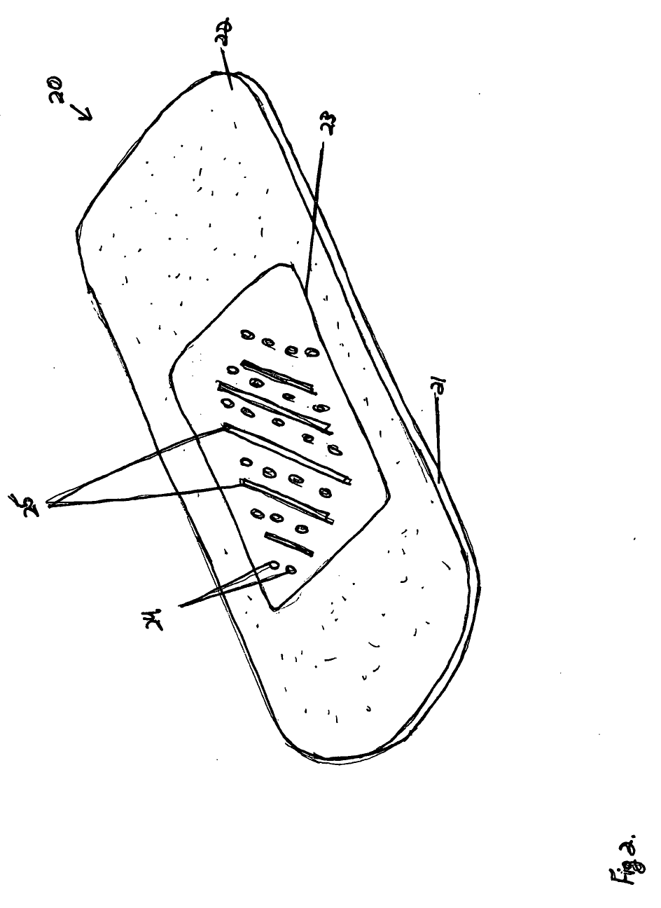

FIG. 2 depicts a second wound dressing in accordance with the present invention. Referring to FIG. 2, the wound dressing 20 comprises a backing 21, an adhesive layer 22, and a region 23 of the backing that does not have an adhesive layer thereon. The adhesive layer 22 is coated on one side of the backing 21. The region 23 has coated thereon at least two salts, 24 and 25, of a sulfonated styrene copolymer in a preselected pattern.

In this example, two salts are shown. Portions of the adhesive layer 22 are exposed and are used to secure the dressing 20 to a body or body part in the same fashion as a conventional adhesive bandage. The first salt 24 was applied onto the region 23 in a preselected pattern of dots. The second salt 25 was applied onto the region 23 in a preselected pattern of stripes. Alternatively the both or either of the salts 24 and 25 could have been impregnated into the region 23.

A method for controlling chemical agents and/or biological organisms is presented in accordance with the present invention. The method comprises providing a composition comprising: i) a substrate and ii) a polymer composition disposed on at least a first side of the substrate, wherein the polymer composition comprises at least two salts of a sulfonated styrene copolymer having a chemical controlling agent and/or biological organism controlling agent as a counter ion in an embodiment of the present invention. The method further comprises exposing said composition to a chemical agent and/or a biological organism wherein said chemical controlling agent and/or said biological organism controlling agent interacts with a chemical agent and/or a biological organism.

The substrate that may be used includes but is not limited to a solid film, a perforated film, a woven fabric, a non-woven fabric, a knit fabric, a latex material, a porous material, a non-porous material, a polymeric material, an article of wear, and the like. Typically the substrate is a wound dressing such as hydrogels, hydrocolloids, gauze, tapes, bandages, and the like.

The polymer composition disposed on the wound dressing comprises at least two salts of the sulfonated styrene copolymer having at least one structural unit represented by the formula 1: 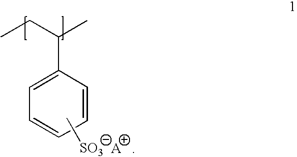
wherein A⁺ is chemical controlling agent such as Co ion, a V ion, a Ru ion, a Pd ion, a Rh ion, a Mn ion, an Fe ion, a Cu ion, a Pt ion, an ion of chloroamine, an ion of hydrazine, an ammonium ion, an ion of an alkyl amine, an ion of an alkyl diamine, an ion of an aromatic amine, an ion of an aromatic diamine. The counter ion A⁺ may also be a biological organism controlling agent such as groups comprising of ions of antibacterial agents, ions of antiviral agents, ions of antiparasitic agents, ions of antifungal agents, ions of thrombogenic agents, and the like.

The sulfonated styrene copolymers typically used in an embodiment of the present invention include but are not limited to a sulfonated styrene-ethylene-butylene-styrene triblock copolymer, a sulfonated styrene-ethylene, copolymer, a sulfonated styrene-acrylate copolymer, a sulfonated styrene-isobutylene-styrene copolymer, and a sulfonated styrene-divinylbenzene copolymer.

The chemical agents that may be controlled by the method of the present invention include but are not limited to Tabun, Sarin, Soman, methylphosphonothioic acid, sulphur mustard, nitrogen mustard, Lewisite, phosgene oximine, phosgene, diphosgene, chlorine, chloropicrin, and the like.

The chemical controlling agents described previously in application are not limited to only controlling the chemical agents listed immediately above. The chemical controlling agents of the present invention also are for controlling enzymes (e.g. elastase, collagonase), enzymatic processes, biological processes (e.g. inflammation and cell growth), and the like. Although the aforementioned enzymes, enzymatic processes, biological processes, and the like might be considered in another classification, we will consider them under the broader classifications of chemical processes and chemical species, i.e. chemical agents.

The biological organisms that may be controlled by the method of the present invention include but are not limited to Anthrax, Botulinum Toxins, Brucellosis, Cholera, Clostridium perfringens Toxins, Congo-Crimean Hemorrhagic fever, Ebola Haemorrhagic fever, Melioidosis, Plague, Q fever, Ricin, Rift Valley fever, Saxitoxin, Smallpox, Staphylococcal Enterotoxin B, Trichothecene Mycotoxins, Tularemia, Venezuelan Equine Encephalitis, and the like.

A wound dressing having the polymer composition, described above and throughout the application, can be used to interact with chemical agents or biological organisms. First one places the side of the wound having the two salts of a sulfonated styrene copolymer with a chemical controlling agent and/or biological organism controlling agent as a counter ion on the wound. A wound is a physical injury to a tissue, or any degradation of its normal structure and function resulting from and internal or external pathology that result in an opening or break of the skin.

The wound dressings of the present invention promote optimum physiological conditions for healing in and/or of the wound by maintaining or promoting tissue hydration. When applied to dry wounds, the dressing rehydrates desiccated tissue, either by preventing loss of water vapor from the site or by directly transferring moisture to the tissue. When applied to exuding wounds, the dressing absorbs the exudate and promotes hydration of the tissue.

Autolytic debridement of necrotic tissue and/or formation of new tissue occur more readily under these conditions. In addition, a variety of growth factors that promote wound healing are present in the exudates from the wounds (see Howell, J. M., Current and Future Trends in Wound Healing, Emerg. Med. Clin. North Amer., 10, 655-663 (1992)), and it is believed that the moist wound dressing can absorb fluid from the exudates. This promotes healing by minimizing loss of these growth factors from the wound bed. After the dressing is applied to the wound, the therapeutic agent or chemical controlling agent diffuses into the tissue where it interacts with any biological organisms or chemical agents present. The interaction may comprise neutralization of activity, destruction of, and/or removal of the biological organisms or chemical agents present.

A process for patterning a wound dressing is presented in accordance with the present invention. The process comprises: providing a substrate; applying a sulfonated styrene copolymer onto said substrate to form a wound dressing; applying a first salt onto said wound dressing in a preselected pattern; and applying a second salt onto said wound dressing in a preselected pattern.

Typical substrates used in an embodiment of the present invention include but are not limited to solid films, porous materials, non-porous materials, polymeric materials, gauzes, tapes, bandages, and the like. A sulfonated styrene copolymer is applied to the substrate forming the wound dressing. Techniques of applying the sulfonated styrene copolymer to the substrate include but are not limited to ink-jet printing techniques, solution casting techniques, dip coating techniques, electroprocessing techniques, web coating techniques, and the like.

The salt then is applied to the wound dressing in a preselected pattern. Salts typically include but are not limited to a base or any agent that causes deprotonation of the sulfonated styrene copolymer or any mechanism that produces a salt of the sulfonated styrene copolymer

Preselected patterns that may be used in an embodiment of the present invention include but are not limited to stripes, circles, lines, squares, dots, and the like. The second salt then is applied onto the first salt in a preselected pattern. Typically, salts that are applied include but are not limited to salts of a chemical controlling agent and/or a salt of biological controlling agent. Application techniques and available patterns are as described above. Two examples of the patterning process for a wound dressing are described below.

A poly(ethylene terephthalate) (PET) fabric was used as substrate and coated with a 60% sulfonated styrene-ethylene-butylene-styrene (SSEBS) triblock copolymer solution to form a wound dressing. The SSEBS coated fabric then was treated with a first salt being NaHCO₃ to deprotonate the sulfonated styrene unit of the SSEBS copolymer affording a first salt of the sulfonated styrene copolymer (—SO₃⁻Na⁺). The coated fabric then was treated with a second salt doxycycline (Dox):HCl. Drops of a Dox:HCl solution (0.1 Molar (M) in dionized (DI) H₂O were placed on the wound dressing in a preselected pattern (dots). The fabric was allowed to dry and then further treated with DI water to rinse away any unbound doxycycline.

The final result was a composition comprising a PET fabric coated with the —SO₃⁻Na⁺ salt and a —SO₃⁻Dox⁺ salt wherein the salts were in a preselected pattern of polka dots across the surface of the PET fabric. Another example of the composition is a PET fabric coated with —SO₃⁻Na⁺ salt and a —SO₃⁻Argenine⁺ salt wherein the —SO₃⁻Arginine⁺ salt was in a polka dot pattern across the surface of the PET fabric. The composition was formed as described above by substituting Sodium with Arginine.

Preselected patterns that may be used in an embodiment of the present invention include but are not limited to stripes, circles, lines, squares, dots, and the like. It can be envisioned that the pattern preselected for application of the first salt may or may not be a pattern that does not fully cover the wound dressing, i.e. areas of the wound dressing do not have any of the first salt applied thereon.

The second salt then is applied onto the wound dressing in areas that do not have a first salt. As with the first salt, application of the second salt is performed using a preselected pattern. Application techniques and available patterns available for use are as described above.

The process for patterning a wound dressing described above is not meant to limit the scope patterning process for wound dressings in an embodiment of the present invention. A patterning process can be envisioned where both salts are applied to the substrate. For example, after a first salt has been applied to a substrate in a preselected pattern, a second salt of the sulfonated styrene copolymer may be applied onto the substrate and/or the wound dressing in a preselected pattern.

The preparation of wound dressings and compositions having at least two salts of a sulfonated styrene copolymer in a preselected pattern thereon in accordance with the present invention were motivated by unexpected results discovered from experimentation. A poly(ethylene terephthalate) (PET) fabric was used as a substrate and coated with a 60% sulfonated styrene-ethylene-butylene-styrene (SSEBS) triblock copolymer solution.

The SSEBS coated fabric then was treated with NaHCO₃ to deprotonate the sulfonated styrene unit of the SSEBS copolymer affording the first salt of the sulfonated styrene copolymer (—SO₃⁻Na⁺). The entire coated fabric then was treated to afford the second salt of the sulfonated styrene copolymer. A Doxycycline (Dox):HCl solution was not applied to the fabric in a preselected pattern. The fabric was allowed to dry and then further treated with DI water to rinse away any unbound Dox. The coated fabric now comprised a —SO₃⁻Na⁺ layer with a layer of—SO₃⁻Dox⁺ salt covering the —SO₃⁻Na⁺ layer in its entirety.

The coated fabric was then tested against MMP-8 and elastase. The —SO₃⁻Na⁺ salt is effective for inhibition of elastase as demonstrated by prior experimentation and was expected to be effective against elastase in this example. But unexpectedly, the —SO₃⁻Na⁺ salt was ineffective for elastase inhibition. The result was further supported by a second experiment substituting the SSEBS coated fabric with a sulfonated SIBS coated fabric and subsequently a SIBS—SO₃⁻Na⁺ salt. The result was again unexpected ineffectiveness of the —SO₃⁻Na⁺ salt for elastase inhibition. In both examples, the doxycycline was effective against MMP-8.

The experimental data indicates that it is necessary to separate the doxycycline and sodium derivatives into separate locations. Any attempt to partially occupy available sites for exchange, i.e. Na⁺ for Dox⁺ by exposure to the entire substrate will result in creating a stiffer dressing as well as eliminating the possibility of creating a dressing with dual/simultaneous anti-protease functionality (i.e. inhibition of MMP-8 and elastase). The two salts must be applied in a preselected pattern to retain the functionality of each salt. The salts may overlap or partially occupy available sites for interaction with a chemical agent or biological organism but not over the entirety of a substrate. Preselected patterning of salts of sulfonated polystyrene copolymers allows for maximum effectiveness of the functionality of the applied salts toward a directed target.

What is claimed is:

1. A composition comprising:
a substrate;
a polymer composition disposed on at least a first side of said substrate, wherein said polymer composition comprises at least two salts of a sulfonated styrene copolymer having a chemical controlling agent and/or a biological organism controlling agent as a counter ion.

2. The composition of claim 1, wherein said substrate is selected from the group consisting of a solid film, a perforated film, a woven fabric, a non-woven fabric, a knit fabric, a latex material, a porous material, a non-porous material, a polymeric material, an article of wear, and combinations thereof.

3. The composition of claim 1, wherein said substrate is a wound dressing.

4. The composition of claim 1, wherein said sulfonated styrene copolymer is selected from the group consisting of a sulfonated styrene-ethylene-butylene-styrene triblock copolymer, a sulfonated styrene-ethylene copolymer, a sulfonated stryrene-acrylate copolymer, a sulfonated styrene-isobutylene-styrene copolymer, a sulfonated styrene-divinylbenzene copolymer, and combinations thereof.

5. The composition of claim 1, wherein said chemical controlling agent includes a counter ion selected from the group consisting of a Cobalt ion, a Vanadium ion, a Ruthenium ion, a Palladium ion, a Rhodium ion, a Manganese ion, an Iron ion, a Copper ion, a Platinum ion, an ion of chloroamine, an ion of hydrazine, an ammonium ion, an ion of an alkyl amine, an ion of an alkyl diamine, an ion of an aromatic amine, an ion of an aromatic diamine, and combinations thereof.

6. The composition of claim 1, wherein said biological organism controlling agent includes an agent selected from the group consisting of an antibacterial agent, an antiviral agent, an antiparasitic agent, an antifungal agent, and combinations thereof.

7. The composition of claim 6, wherein said antibacterial agent is selected from the group consisting of an ion of chlorhexidine, an ammonium ion, a ion of benzalkyl amine, a hexyl pyridinium ion, an ion of an alkyl amine, a Cobalt ion, a Vanadium ion, a Ruthenium ion, a Palladium ion, a Rhodium ion, a Manganese ion, an Iron ion, a Copper ion, a Platinum ion, and combinations thereof.

8. The composition of claim 6, wherein said antiviral agent is selected from the group consisting of an ion of acyclovir, an ion of trifluorouridine, an ion of foscarnet, an ion of ganciclovir, and combinations thereof.

9. The composition of claim 6, wherein said antiparasitic agent is selected from the group consisting of nystatin, metronidazole, tinidazole, diloxanide, and combinations thereof.

10. The composition of claim 5, wherein said antifungal agent is selected from the group consisting of an allylamine, an azole, an antimetabolite, a glucan synthesis inhibitor, and combinations thereof.

11. The composition of claim 1, wherein said polymer composition comprises a sulfonated styrene-alkali metal salt and a sulfonated styrene-therapeutic agent salt.

12. The composition of claim 1, wherein one sulfonated styrene salt of said polymer composition is a sulfonated styrene-alkylene salt having a Calcium ion, a Zinc ion, a Manganese ion, a Magnesium ion, an ion of lysine, an ion of polylysine, an ion of aminobutryic acid, or an ion of aminohexanoic acid as said counter ion.

13. The composition of claim 1, wherein one of said at least two salts is lipophilic and the other is hydrophilic.

14. The composition of claim 1, wherein a first salt is in a ratio to a second salt in a range from about 0.1 mole % said first salt to 99.9 mole % said second salt to about 99.9 mole % said first salt to about 0.1 mole % said second salt.

15. A wound dressing comprising:
at least a first layer comprising a first salt of a sulfonated styrene copolymer having at least one surface contactable with a wound and disposed on said one surface a second salt of said sulfonated styrene copolymer, wherein said second salt is disposed on said one surface in a preselected pattern.

16. The wound dressing according to claim 15, wherein one structural unit of said first salt of said sulfonated styrene copolymer or said second salt of said sulfonated styrene copolymer is represented by a formula 1: 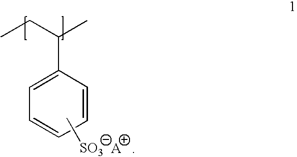
wherein A⁺ is an alkali metal ion, an alkaline earth ion, an oligodynamic metal ion, a pyridinium ion, a tetra-alkyl ammonium ion, an alkyl ammonium ion, an ion of a benzalkyl amine, an alkyl pyrazinium ion, an ion of an alkyl amine, or an ion of a chlorhexidine.

17. The wound dressing according to claim 15, wherein one structural unit of said first salt of said sulfonated styrene copolymer or said second salt of said sulfonated styrene copolymer is represented by a formula 1: 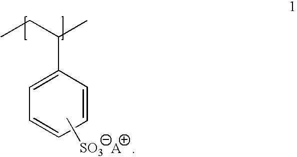
wherein A⁺ is a Hydrogen ion, an ammonium ion, an ion of an alkali metal, an ion of an oligodynamic metal, or a therapeutic agent.

18. The wound dressing according to claim 17, wherein said therapeutic agent is selected from the group consisting of an antibacterial agent, an anesthetic, a growth factor, spermicide, an antiviral agent, an anti-inflammatory agent, an antihistamine, an analgesic agent, an anti-neoplastic agent, an antihistamine, a hormone, a kerolytic agent, a tranquilizer, a vitamin, a base-pair nucleotide, a cytokine, and combinations thereof.

19. The wound dressing according to claim 15, wherein one structural unit of said first salt of said sulfonated styrene copolymer or said second salt of said sulfonated styrene copolymer is represented by a formula 1: 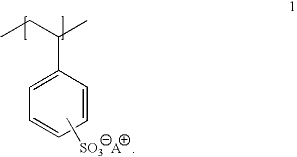
wherein A⁺ is a Cobalt ion, an ion comprising Vanadium, a Ruthenium ion, a Palladium ion, an Iron ion, a Manganese ion, a Platinum ion, a Rhodium ion, a Copper ion, an ion of chloroamine, an ion of hydrazine, an ion of an alkyl diamine, an ion of an aromatic amine, an ion of an aromatic diamine ion, and combinations thereof.

20. The wound dressing of claim 15, wherein said sulfonated styrene polymer is a sulfonated styrene-ethylene-butylene-styrene triblock copolymer.

21. A wound dressing comprising:
at least a first layer impregnated with at least two salts of a sulfonated styrene copolymer having at least one surface contactable with a wound, wherein said at least two salts are impregnated in said first layer in a preselected pattern.

22. The wound dressing according to claim 21, wherein one structural unit of said first salt of said sulfonated styrene copolymer or said second salt of said sulfonated styrene copolymer is represented by a formula 1: 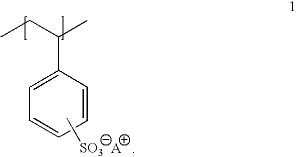
wherein A⁺ is an alkali metal ion, an alkaline earth ion, an oligodynamic metal ion, an alkyl pyridinium ion, a tetra-alkyl ammonium ion, an alkyl ammonium ion, an ion of a benzalkyl amine, an alkyl pyrazinium ion, an ion of an alkyl amine, or an ion of chlorhexidine.

23. The wound dressing according to claim 21, wherein one structural unit of said first salt of said sulfonated styrene copolymer or said second salt of said sulfonated styrene copolymer is represented by a formula 1: 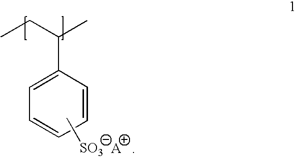
wherein A⁺ is a Hydrogen ion, an ammonium ion, an ion of an alkali metal, an ion of an oligodynamic metal, or a therapeutic agent.

24. The wound dressing according to claim 21, wherein said therapeutic agent is selected from the group consisting of an antibacterial agent, an anesthetic, a growth factor, spermicide, an antiviral agent, an anti-inflammatory agent, an antihistamine, an analgesic agent, an anti-neoplastic agent, an antihistamine, a hormone, a kerolytic agent, a tranquilizer, a vitamin, a base-pair nucleotide, a cytokine, and combinations thereof.

25. The wound dressing according to claim 21, wherein one structural unit of said first salt of said sulfonated styrene copolymer or said second salt of said sulfonated styrene copolymer is represented by a formula 1: 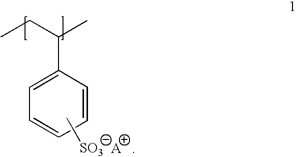
wherein A⁺ is a Cobalt ion, an ion comprising Vanadium, a Ruthenium ion, a Palladium ion, an Iron ion, a Manganese ion, a Platinum ion, a Rhodium ion, a Copper ion, an ion of a chloroamine, an ion of a hydrazine, an ion of an alkyl diamine ion, an ion of an aromatic amine, an ion of an aromatic diamine, and combinations thereof.

26. The wound dressing of claim 21, wherein said sulfonated styrene polymer is a sulfonated styrene-ethylene-butylene-styrene triblock copolymer.

27. A method for controlling chemical agents and/or biological organisms comprising:
providing a composition comprising: i) a substrate and ii) a polymer composition disposed on at least a first side of said substrate, wherein said polymer composition comprises at least two salts of a sulfonated styrene copolymer having a chemical controlling agent and/or a biological organism controlling agent as a counter ion; and
exposing said composition to a chemical agent and/or a biological organism wherein said chemical controlling agent and/or said biological organism controlling agent interacts with a chemical agent and/or a biological organism.

28. The method of claim 27, wherein said sulfonated styrene copolymer is a sulfonated styrene-ethylene-butylene-styrene triblock copolymer, a sulfonated styrene-ethylene, copolymer, a sulfonated styrene-acrylate copolymer, a sulfonated styrene-isobutylene-styrene copolymer, a sulfonated styrene-divinylbenzene copolymer, and combinations thereof.

29. The method of claim 27, wherein said chemical controlling agent is selected from the group comprising a Cobalt ion, an ion comprising Vanadium, a Ruthenium ion, a Palladium ion, an Iron ion, a Manganese ion, a Platinum ion, a Rhodium ion, a Copper ion, a chloroamine ion, a hydrazine ion, an alkyl diamine ion, an aromatic amine ion, an aromatic diamine ion, and combinations thereof.

30. The method of claim 27, wherein said biological organism controlling agent is selected from the group comprising an antibacterial agent, an antiviral agent, an antiparasitic agent, an antifungal agent, and compositions thereof.

31. The method of claim 27, wherein said chemical agent is selected from the group consisting of Tabun, Sarin, Soman, methylphosphonothioic acid, sulphur mustard, nitrogen mustard, Lewisite, phosgene oximine, phosgene, diphosgene, chlorine, chloropicrin, and combinations thereof.

32. The method of claim 27, wherein said biological organism is selected from the group consisting of Anthrax, Botulinum Toxins, Brucellosis, Cholera, Clostridium perfringens Toxins, Congo-Crimean Hemorrhagic fever, Ebola Haemorrhagic fever, Melioidosis, Plague, Q fever, Ricin, Rift Valley fever, Saxitoxin, Smallpox, Staphylococcal Enterotoxin B, Trichothecene Mycotoxins, Tularemia, Venezuelan Equine Encephalitis, and combinations thereof.

33. A process for patterning a wound dressing comprising:
providing a substrate;
applying a sulfonated styrene copolymer to said substrate to form a wound dressing;
applying a first salt onto said wound dressing in a preselected pattern; and
applying a second salt onto said wound dressing in a preselected pattern.

34. The process for patterning a wound dressing according to claim 33, wherein said substrate is selected from the group consisting of a solid film, a perforated film, a woven fabric, a non-woven fabric, a knit fabric, a latex material, a porous material, a non-porous material, a polymeric material, an article of wear, and combinations thereof.

35. The process for patterning a wound dressing according to claim 33, wherein said applying is performed by techniques selected from the group comprising of ink-jet printing techniques, solution casting techniques, dip coating techniques, electroprocessing techniques, web coating techniques, and combinations thereof.

36. The process for patterning a wound dressing according to the claim 33, wherein said first salt is any reagent capable of producing a salt of said sulfonated styrene copolymer having one structural unit represented by formula 1: 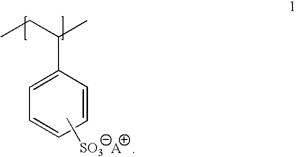
upon application of said first salt to said wound dressing.

37. The process for patterning a wound dressing according to claim 33, wherein said preselected pattern is selected from the group consisting of stripes, circles, lines, squares, dots, and combinations thereof.
